(19) United States
(12) Patent Application Publication
(10) Pub. No.: US 2008/0108991 A1
(43) Pub. Date: May 8, 2008

(54) METHOD AND APPARATUS FOR PERFORMING PEDICLE SCREW FUSION SURGERY

(75) Inventor: Ron Andrew von Jako, Melrose, MA (US)

Correspondence Address: PETER VOGEL;GE HEALTHCARE, 3000 N. GRANDVIEW BLVD., SN-477, WAUKESHA, WI, 53188, US

(73) Assignee: General Electric Company, Schenectady, NY (US)

(21) Appl. No.: 11/557,582

(22) Filed: Nov. 8, 2006

(51) Int. Cl. A61B 17/70

(52) U.S. Cl. 606/61

(57) ABSTRACT

A medical device adapted to facilitate pedicle screw fusion surgery is disclosed herein. The medical device includes a proximal end and a sharp distal end opposite the proximal end. The distal end is configured to allow the medical device to function as an awl. The medical device also includes a body portion defined between the proximal end and the distal end, and a threaded section defined by the body portion near the distal end. The threaded section is configured to allow the medical device to function as a tap. Accordingly, the medical device provides a single tool adapted to function as both an awl and a tap. A corresponding method for securing a pedicle screw to a vertebra is also provided.

### FIELD OF THE INVENTION

This invention pertains generally to a method and apparatus for performing pedicle screw fusion surgery.

### BACKGROUND OF THE INVENTION

Pedicle screw fusion surgery generally involves the insertion of pedicle screws into a short tubular structure connecting the vertebral body with the lamina and represents the strongest portion of the vertebra found on each side of the vertebra. This allows the pedicle screws to grab into the bone of the vertebral body, giving them a solid hold on the vertebra. Once the pedicle screws are placed, they are attached to metal rods that connect the screws together. This creates a stiff metal frame that holds the vertebrae still and thereby facilitates decompression for pain relief and healing. Bone graft is typically placed around the back of the vertebra to help the vertebrae heal together, or fuse.

Pedicle screw fusion procedures can incorporate surgical navigation technology wherein the location of a medical device is measured and virtually superimposed on a patient image. The patient image may be pre-recorded, near real-time, or real-time, and is preferably obtained using known imaging technology such as X-ray, computed tomography (CT), magnetic resonance imaging (MRI), or ultrasound (US). Conventional navigation technology measures the location of a remote unit attached to the medical device relative to a reference unit. Patient motion can be taken into account by rigidly mounting the reference unit directly onto the patient. A reference unit attached in this manner is also referred to as a dynamic reference because it moves along with the patient.

A number of medical instruments including an awl, a blunt probe, a ball tip probe-feeler, and a tap are commonly implemented during pedicle screw fusion surgery. Awls have a sharp pointed end that is used to create a shallow pilot hole opening through the bone surface. The pointed end of the awl is particularly well adapted to pedicle screw fusion surgery as blunt instruments would be prone to sliding off the pedicle's dome shaped surface. The blunt probe is used to carve the hole from the pedicle cortex into the vertebal body at an appropriate angle and depth. The ball tip probe-feeler is implemented to verify pedicle integrity via a process of palpation and thereby ensure no violation of the pedicels walls occurred prior to implantation. If a non-significant violation is detected, the pedicle screw is either redirected or the site is abandoned. A tap includes a cutting edge adapted to form internal threads in the cancellous bone of the pedicle canal. The internal treads formed by the tap engage complementary external pedicle screw threads to retain and secure the pedicle screws. These taps that are specifically matched to implant screw counterparts, are commonly used after the pedicle hole is created and prior to implant screw placement.

Conventional awls, blunt probes and taps are individual medical instruments adapted to perform a specialized function as previously described. These individual instruments must be switched back and forth many times during the course of a single pedicle screw fusion procedure. Though these instruments are necessary for the conventional technique, the intra-operative steps associated with switching the instruments are a potential source of inefficiency and can prolong the overall duration of a procedure.

### SUMMARY OF THE INVENTION

The above-mentioned shortcomings, disadvantages and problems are addressed herein which will be understood by reading and understanding the following specification.

In an embodiment, a medical device adapted to facilitate pedicle screw fusion surgery includes a proximal end and a sharp distal end opposite the proximal end. The distal end is configured to allow the medical device to function as an awl. The medical device also includes a body portion defined between the proximal end and the distal end, and a threaded section defined by the body portion near the distal end. The threaded section is configured to allow the medical device to function as a tap. Accordingly, the medical device provides a single tool adapted to function as both an awl and a tap.

In another embodiment, a system adapted to facilitate pedicle screw fusion surgery includes a position detection process in communication with a remote unit and a reference unit. The position detection process is configured to estimate the location of the remote unit relative to the reference unit. The system also includes a medical device attached to the remote unit. The medical device includes a proximal end, a distal end, and a body portion defined therebetween. The medical device also includes a generally pointed tip defined at the distal end. The pointed tip is configured to allow the medical device to function as an awl. The medical device also includes a threaded section defined by the body portion near the distal end. The threaded section is configured to allow the medical device to function as a tap. The system also includes a display operatively connected to the position detection device. The display is adapted to convey the location of the medical device relative to the reference unit.

In yet another embodiment, a method for securing a pedicle screw to a vertebra includes creating a pilot hole in a pedicle with a medical device, forming a screw hole in the vertebra at the location of the pilot hole with the medical device, forming an internal thread disposed about the periphery of the screw hole with the medical device, and inserting an externally threaded pedicle screw into the internally threaded screw hole. Wherein creating a pilot hole, forming a screw hole, and forming an internal thread are all performed with a single medical device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a navigation system;

FIG. 2 is a detailed perspective illustration of a medical device in accordance with an embodiment;

FIG. 2a is a detailed perspective illustration of the distal end of the medical device of FIG. 2 in accordance with an embodiment;

FIG. 2b is a detailed perspective illustration of the distal end of the medical device of FIG. 2 in accordance with an embodiment;

FIG. 3a is a cross sectional view of a vertebra having a pilot hole formed in each pedicle;

FIG. 3b is a cross sectional view of a vertebra having a screw hole formed in each pedicle;

FIG. 3c is a cross sectional view of a vertebra having an internally threaded hole formed in each pedicle;

FIG. 3d is a cross sectional view of a vertebra having a pedicle screw inserted into each pedicle; and

FIG. 4 is a block diagram illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

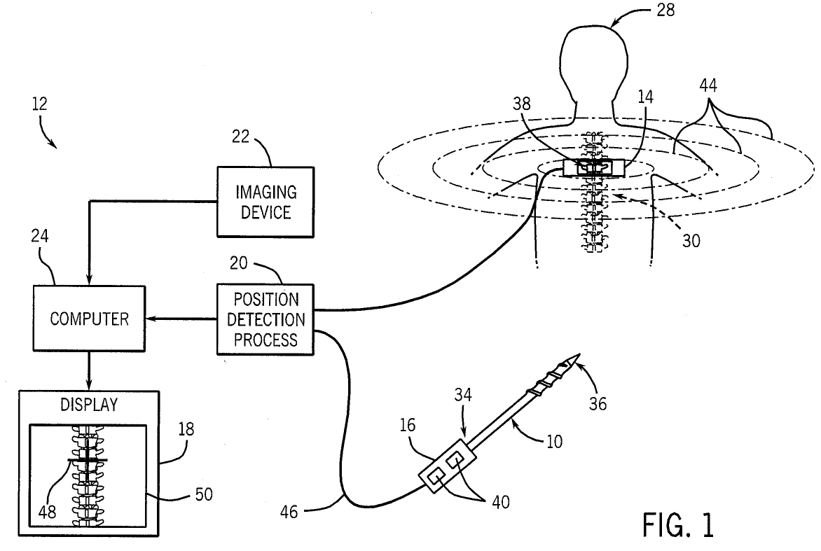

Referring to FIG. 1, a medical device or instrument 10 in accordance with an embodiment is shown. The medical device 10 may be implemented with a variety of different navigation systems such as, for example, the surgical navigation system 12. The navigation system 12 includes a reference unit 14, a remote unit 16, a display 18, a position detection process 20, an imaging device 22 and a computer 24.

The reference unit 14 is preferably rigidly attached to a patient 28 near the target operation site 30 (e.g., a portion of the spine) in a conventional manner. A reference unit attached in this manner is also referred to as a “dynamic reference” because it moves along with the patient. The remote unit 16 is attached to the medical device 10. The present invention will hereinafter be described in accordance with an embodiment wherein the reference unit 14 includes a field generator 38, and the remote unit 16 includes one or more field sensors 40. It should, however, be appreciated that according to alternate embodiments the reference unit may include the field sensors and the remote unit may include the field generator.

The field generator 38 in the reference unit 14 generates a position characteristic field 44 in an area that includes the target operation site 30. The field sensors 40 in the remote unit 16 produce sensor signals (not shown) in response to the sensed position characteristic field 44. The sensor signals are transmitted or input into the position detection process 20. The sensor signals may be transmitted via communication line 46, or may be wirelessly transmitted. The position detection process 20 is adapted to estimate the location of the remote unit 16 relative to the reference unit 14. A known calibration procedure can be implemented to estimate the location of the distal end or tip 36 of the medical device 10.

The location of the medical device 10 may be conveyed via the display 18. According to one embodiment, a graphical representation 48 of the distal end 36 is virtually superimposed onto a patient image 50. More precisely, the graphical representation 48 of the distal end 36 is virtually superimposed onto the portion of the image 50 that corresponds to the actual location of the distal end 36 within the patient 28. The graphical representation 48 may include a dot or cross hairs identifying just the distal end 36, or may include a more complete rendering showing the medical device 10 in detail.

According to one embodiment, the patient image 50 is obtained prior to the medical procedure using known imaging technology such as X-ray, computed tomography (CT), magnetic resonance imaging (MRI), or ultrasound (US). Additionally, during the course of the medical procedure, the imaging device 22 may be implemented to observe the patient 28 in real-time or near real-time. Therefore, the pre-recorded patient image 50 can be replaced with a real-time patient image or a near real-time image as desired. According to an exemplary embodiment, the imaging device 22 may include a fluoroscopic X-ray device mounted to a C-arm, however, other imaging devices may also be implemented.

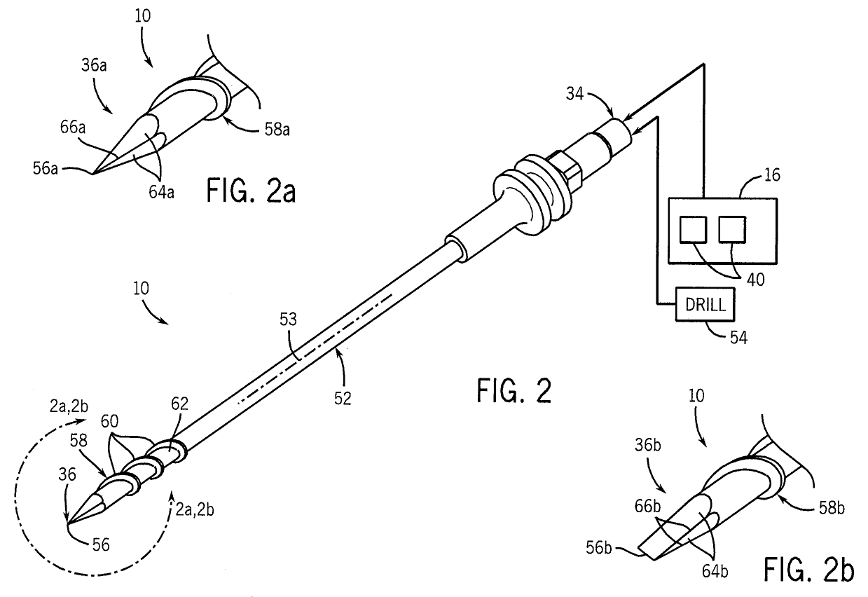

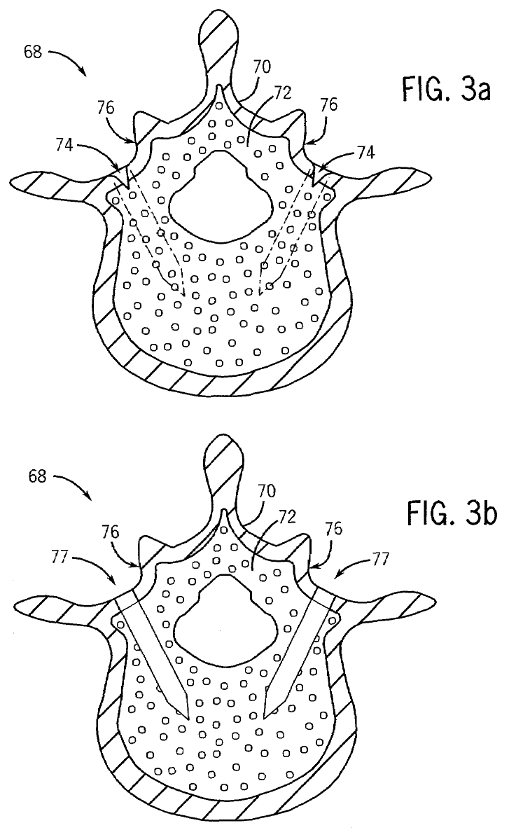

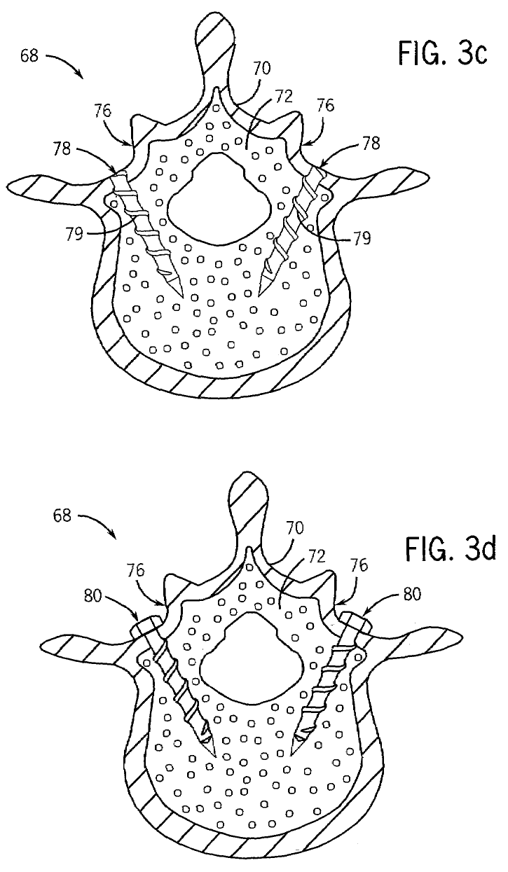

Referring to FIG. 2, the medical device 10 is shown in more detail. The medical device 10 is preferably comprised of a material compatible with electro-magnetic tracking technology such a non-metallic material or a minimally conductive metal so that position characteristic field 44 (shown in FIG. 1) is not distorted. The medical device 10 includes a proximal end 34, the distal end 36, and a generally cylindrical body portion 52 defined therebetween. The generally cylindrical body portion 52 defines an axis 53. The proximal end 34 of the medical device 10 is adapted to receive the remote unit 16 and/or a driver such as, for example, the power drill 54. The body portion 52 tapers to a sharp point 56. The point 56 is similar to the tip of a conventional awl (not shown). The point 56 is configured to pierce through cortical bone and into cancellous bone in order to produce a pilot hole 74 (shown in FIG. 3a).

The body portion 52 of the medical device 10 defines a threaded section 58 near the distal end 36. The threaded section 58 is similar to that of a conventional tap (not shown), and includes one or more external threads 60 defining a cutting edge 62. The cutting edge 62 is configured to cut both cortical bone and cancellous bone, and thereby form an internally threaded hole 78 (shown in FIG. 3c) adapted to accommodate a pedicle screw 80 (shown in FIG. 3d).

Referring to FIG. 2a, a distal end 36a configuration is shown in accordance with one embodiment. The distal end 36a of the medical device 10 defines a plurality of generally flat sections 64a disposed axially between the point 56a and the threaded section 58a. For purposes of this disclosure, the terms “flat section” and “blunt section” may be used interchangeably. An edge 66a is defined at the intersection of adjacent blunt sections 64a. The blunt sections 64a and the edges 66a are adapted to allow the medical device 10 to function similarly to a conventional blunt probe (not shown). More precisely, by rotating the body portion 52 (shown in FIG. 2) back and forth about its axis 53 (shown in FIG. 2), the edges 66a can carve a screw hole 77 (shown in FIG. 3b) through the pedicle 76 (shown in FIGS. 3a-3d) into the vertebra 68 (shown in FIGS. 3a-3d).

Referring to FIG. 2b, a distal end 36b configuration is shown in accordance with another embodiment. In this embodiment, the body portion 52 (shown in FIG. 2) tapers to a sharp edge 56b rather than the previously described sharp point 56a (shown in FIG. 2a). The distal end 36b of the medical device 10 defines a plurality of blunt sections 64b disposed axially between the edge 56b and the threaded section 58b. An edge 66b is defined at the intersection of adjacent blunt sections 64b. The blunt sections 64b and the edges 66b are adapted to allow the medical device 10 to function similarly to a conventional blunt probe (not shown). More precisely, by rotating the body portion 52 back and forth about its axis 53 (shown in FIG. 2), the edges 66b can carve a screw hole 77 (shown in FIG. 3b) through the pedicle 76 (shown in FIGS. 3a-3d) into the vertebra 68 (shown in FIGS. 3a-3d).

FIGS. 3a-3d, illustrate a sequence of procedures that may be performed by the medical device 10 during pedicle screw fusion surgery. Referring to FIG. 3a, a cross sectional view of a vertebra 68 is shown. The vertebra 68 includes an outer layer of cortical bone 70 surrounding a core of porous cancellous bone 72. A pilot hole 74 is preferably pierced into each pedicle 76 by the point 56 of the medical device 10 (shown in FIG. 2). Referring to FIG. 3b, a screw hole 77 is carved into the vertebra 68 at the location of the pilot holes 74. The screw holes 77 can be formed with the edges 66a (shown in FIG. 2a) as the body portion 52 (shown in FIG. 2) is rotated back and forth about its axis 53 (shown in FIG. 2).

Referring to FIG. 3c, an internal thread 79 is formed in the portion of the vertebra 68 defining the screw hole 77 (shown in FIG. 3b). The internal thread 79 is therefore disposed about the periphery of the screw hole 77 such that an internally threaded hole 78 is produced. The internal thread 79 can be formed by the threaded section 58 of the medical device 10. Referring to FIG. 3d, a pedicle screw 80 is driven into each of the internally threaded holes 78. Metal rods (not shown) connect the pedicle screws 80 of adjacent vertebra together such that the vertebrae are held still. Bone graft (not shown) may be placed around the back of the vertebra to help the vertebrae heal together, or fuse.

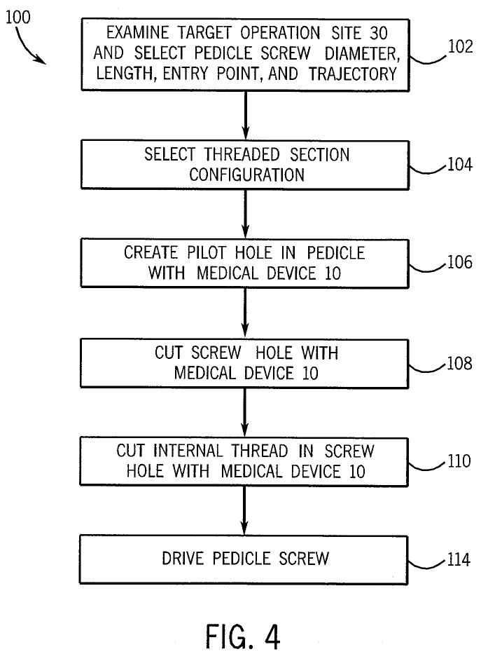

Referring to FIG. 4, a block diagram illustrates a method 100. The individual blocks 102-114 represent steps that may be performed in accordance with the method 100. It should be appreciated that the steps of the method 100 described in detail hereinafter are preferably performed in combination with a navigation system such as the surgical navigation system 12 (shown in FIG. 1).

At step 102, the target operation site 30 (shown in FIG. 1) is examined to select an optimal pedicle screw diameter, length, entry point and trajectory. The imaging device 22 (shown in FIG. 1) is preferably implemented during this step to directly observe the pedicle 76 (shown in FIGS. 3a-3d) and thereby provide a more accurate estimate. An imaging device 22 implementing fluoroscopic imaging technology is particularly well adapted for use during step 102.

At step 104, an optimal configuration for the threaded section 58 of the medical device 10 (shown in FIG. 2) is selected. More precisely, the threaded section 58 configuration is selected to produce an internally threaded hole 78 (shown in FIG. 3c) that is sized to receive a pedicle screw 80 (shown in FIG. 3d) having predetermined selectable features such as screw diameter, length, thread density, etc. The diameter of the internally threaded hole 78 may be formed to approximately match that of the pedicle screw 80, or may be somewhat undersized to produce an interference fit or crush fit technique. For purposes of the present invention, an “nterference fit technique” is one wherein a pedicle screw is forcibly driven into an undersized or relatively smaller pedicle hole such that, during insertion, the screw is compressed by the pedicle bone. The compressive force applied by the pedicle bone to the pedicle screw is intended to improve screw retention.

At step 106, the medical device point 56 is implemented to pierce the pedicle 76 (shown in FIGS. 3a-3d) at the pre-selected entry point such that a pilot hole 74 (shown in FIG. 3a) is created. The navigation system 12 (shown in FIG. 1) is preferably implemented to guide the medical device 10 (shown in FIG. 2) and thereby ensure that the pilot hole 74 is precisely located at the pre-selected entry point. Advantageously, implementing the navigation system 12 to guide the medical device 10 obviates the need for a guide-wire (not shown) that would otherwise be required for locating the pedicle entry point. Therefore, the method 100 expedites the entire medical procedure by eliminating the time associated with inserting and placing a guide-wire. Conventional targeting software may also be implemented at this step to help guide the medical device 10 toward the pre-selected entry point.

At step 108, the screw holes 77 (shown in FIG. 3b) are formed. More precisely, while the distal end 36 (shown in FIG. 2) remains disposed within the pilot hole 74 (shown in FIG. 3a), the body portion 52 (shown in FIG. 2) is rotated back and forth about its axis 53 (shown in FIG. 2). During this step, the axis 53 should remain aligned with the pre-selected pedicle screw trajectory in order to properly form the screw holes 77.

At step 110, the threaded section 58 of the medical device 10 (shown in FIG. 2) is introduced into the screw hole 77 (shown in FIG. 3b) in alignment with the pre-selected pedicle screw trajectory, and thereafter the medical device 10 is translated toward the pedicle 76 (shown in FIGS. 3a-3d) and generally simultaneously rotated in order to cut the internal threads 79 (shown in FIG. 3c). The medical device 10 may be manually rotated or may be attached to the power drill 54 (shown in FIG. 2) configured to drive the medical device rotation.

At step 114, a pedicle screw 80 (shown in FIG. 3d) is driven into the internally threaded hole 78 (shown in FIG. 3c). The pedicle screw 80 is driven in a conventional manner, such as with a screwdriver. According to an alternate embodiment, the pedicle screw 80 may be a self tapping screw driven directly into the pilot hole 74 (shown in FIG. 3a) or into the screw hole 79 (shown in FIG. 3b) such that step 110 is not required.

Steps 102-114 are repeatable at other sights to place additional pedicle screws 80. Advantageously, step 102-110 can all be performed with a single device (i.e., the medical device 10). Performing these steps in a more conventional manner would require at least three separate tools (i.e., an awl; a probe; and a tap), and the surgeon would have to switch back and forth between these separate tools throughout the procedure. It can therefore be seen that the method 100 which implements a single medical device 10 to perform a variety of different procedures saves the time otherwise required for switching and positioning new instruments during the course of the procedure.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A medical device adapted to facilitate pedicle screw fusion surgery, said medical device comprising:
a proximal end;
a sharp distal end opposite the proximal end, said distal end configured to allow the medical device to function as an awl;
a body portion defined between the proximal end and the distal end; and
a threaded section defined by the body portion near the distal end, said threaded section configured to allow the medical device to function as a tap;
wherein the medical device provides a single tool adapted to function as both an awl and a tap.

2. The medical device of claim 1, wherein the sharp distal end is generally pointed.

3. The medical device of claim 1, wherein the sharp distal end defines an edge.

4. The medical device of claim 1, wherein said proximal end is configured to receive a remote unit for surgical navigation.

5. The medical device of claim 1, wherein said proximal end is configured to receive a power drill.

6. The medical device of claim 1, wherein said medical device is comprised of a material that is compatible with electromagnetic tracking technology.

7. The medical device of claim 1, further comprising a plurality of generally flat sections defined axially between the distal end and the threaded section, and a plurality of edges defined at the intersection of adjacent flat sections.

8. The medical device of claim 7, wherein said plurality of flat sections and said plurality of edges are configured to allow the medical device to function as a probe such that the medical device provides a single tool adapted to function as an awl, a probe, and a tap.

9. A system adapted to facilitate pedicle screw fusion surgery, said system comprising:
a position detection process in communication with a remote unit and a reference unit, said position detection process configured to estimate the location of the remote unit relative to the reference unit;
a medical device attached to the remote unit, said medical device comprising:
a proximal end, a distal end, and a body portion defined therebetween;
a generally pointed tip defined at the distal end, said pointed tip configured to allow the medical device to function as an awl; and
a threaded section defined by the body portion near the distal end, said threaded section configured to allow the medical device to function as a tap; and
a display operatively connected to the position detection device, said display adapted to convey the location of the medical device relative to the reference unit.

10. The system of claim 9, wherein said medical device is comprised of a material that is compatible with electromagnetic tracking technology.

11. The system of claim 9, further comprising a plurality of generally flat sections defined axially between the generally pointed tip and the threaded section, and a plurality of edges defined at the intersection of adjacent flat sections.

12. The system of claim 11, wherein said plurality of generally flat sections and said plurality of edges are configured to allow the medical device to function as a probe such that the medical device provides a single tool adapted to function as an awl, a probe, and a tap.

13. The system of claim 9, wherein said reference unit is a dynamic reference unit rigidly attached to a patient.

14. The system of claim 9, further comprising an imaging device operatively connected to the computer, said imaging device configured to provide real-time or near real-time images of a patient.

15. A method for securing a pedicle screw to a vertebra comprising:
creating a pilot hole in a pedicle with a medical device;
forming a screw hole in the vertebra at the location of the pilot hole with the medical device;
forming an internal thread disposed about the periphery of the screw hole with the medical device; and
inserting an externally threaded pedicle screw into the internally threaded screw hole;
wherein said creating a pilot hole, said forming a screw hole, and said forming an internal thread are all performed with a single medical device.

16. The method of claim 15, wherein said creating a pilot hole, said forming a screw hole, and said forming an internal thread include implementing a surgical navigation system.

17. The method of claim 15, wherein said creating a pilot hole includes implementing targeting software.

18. The method of claim 15, wherein said forming a screw hole includes forming an undersized screw hole to provide an interference fit with a subsequently inserted pedicle screw.
